(19) United States
(12) Patent Application Publication
(10) Pub. No.: US 2026/0336853 A1
(43) Pub. Date: Oct. 29, 2026

(54) STERILIZATION APPARATUS, PACKAGING MACHINE HAVING A STERILIZATION APPARATUS AND A METHOD FOR STERILIZING

(71) Applicant: Tetra Laval Holdings & Finance S.A., Pully (CH)

(72) Inventors: Andrea DONATI, Modena (IT); Eugenio SIGHINOLFI, Modena (IT); Luca POPPI, Modena (IT); Filippo FERRARINI, Modena (IT); Davide VERNAZZA, Modena (IT); Enrico GASPARINI, Modena (IT)

(73) Assignee: Tetra Laval Holdings & Finance S.A., Pully (CH)

(21) Appl. No.: 19/110,851

(22) PCT Filed: Nov. 6, 2023
(86) PCT No.: PCT/EP2023/080760

§ 371 Date: Mar. 12, 2025

(30) Foreign Application Priority Data
Nov. 10, 2022 (IT) 102022000023226

(51) Int. Cl. B65B 55/08; A61L 2/087; A61L 2/186; A61L 2/24; A61L 2/26; A61L 103/00; B65B 9/207; B65B 41/16; B65B 55/10; B65B 57/04; B65B 57/18

(52) U.S. Cl. CPC B65B 55/08; A61L 2/087; A61L 2/186; A61L 2/24; A61L 2/26; B65B 9/207; B65B 41/16; B65B 55/103; B65B 57/04; B65B 57/18; A61L 2103/23; A61L 2202/14

(57) ABSTRACT

There is described a sterilization apparatus for sterilizing a web of packaging material advancing along a web advancement path. The sterilization apparatus comprises:
a first sterilizing device configured to sterilize at least a first face of the web advancing through the first sterilizing device,
a second sterilizing device,
a monitoring device, configured to monitor operation of the first sterilizing device,
a movement sensor configured to monitor advancement of the web along said advancement path, and
a control unit, connected to the monitoring device and the movement sensor, the control unit configured to, if an anomaly is detected in operation of the first sterilizing device:
   interrupt advancement of the web,
   identify a distance traveled by the web between detection of the anomaly and interruption of advancement, and
   sterilize at least a portion of the web by means of the second sterilizing device, the portion of the web being associated to said traveled distance.

### TECHNICAL FIELD

The present invention relates to a sterilization apparatus for sterilizing a web of packaging material, in particular a web of packaging material for the production of sealed packages of a pourable product, in particular a pourable food product.

The present invention also relates to a packaging machine for producing sealed packages of a pourable product, in particular a pourable food product, having a sterilization apparatus.

The present invention also relates to a method for sterilizing a web of packaging material, in particular a web of packaging material for the production of sealed packages of a pourable product, in particular a pourable food product.

### BACKGROUND ART

As is known, many liquid or pourable food products, such as fruit juice, UHT (ultra-high-temperature treated) milk, wine, tomato sauce, etc., are sold in packages made of sterilized packaging material.

A typical example is the parallelepiped-shaped package for liquid or pourable food products known as Tetra Brik Aseptic (registered trademark), which is made by sealing and folding laminated strip packaging material. The packaging material has a multilayer structure comprising a base layer, e.g. of paper, covered on both sides with layers of heat-seal plastic material, e.g. polyethylene. In the case of aseptic packages for long-storage products, such as UHT milk, the packaging material also comprises a layer of oxygen-barrier material (an oxygen-barrier layer), e.g. an aluminum foil, which is superimposed on a layer of heat-seal plastic material, and is in turn covered with another layer of heat-seal plastic material forming the inner face of the package eventually contacting the food product.

Packages of this sort are normally produced on fully automatic packaging machines, which advance a web of packaging material from a magazine unit through a sterilization apparatus for sterilizing the web of packaging material and to an isolation chamber (a closed and sterile environment) in which the sterilized web of packaging material is maintained and advanced. During advancement of the web of packaging material through the isolation chamber, the web of packaging material is folded and sealed longitudinally to form a tube having a longitudinal Seam portion, which is further fed along a vertical advancing direction.

In order to complete the forming operations, the tube is filled with a sterilized or sterile-processed pourable product, in particular a pourable food product, and is transversally sealed and subsequently cut along equally spaced transversal cross sections within a package forming unit of the packaging machine during advancement along the vertical advancing direction.

Pillow packages are so obtained within the packaging machine, each pillow package having a longitudinal sealing band, a top transversal sealing band and a bottom transversal sealing band.

In the recent years, sterilization apparatuses have become available, which are configured to sterilize the web of packaging material by means of the application of physical irradiation, in particular electromagnetic irradiation, even more particular electron beam irradiation. In alternative, solutions are known of sterilization apparatus exploiting hydrogen peroxide, wherein the web of packaging material is sterilized by means of the introduction thereof in a hydrogen peroxide bath.

Even though known sterilization apparatus and, accordingly, also the packaging machine provide for good results, a desire is felt to improve performance of the sterilization apparatus.

A need is felt for efficiently and timely dealing with anomalies in operation of the sterilization apparatus, e.g. anomalies in temperature of a peroxide bath in case of hydrogen peroxide sterilization, an anomaly in radiation of UV lamps and/or an electric arc event in case of sterilization by means of an electron beam.

These anomalies result in a temporary interruption of production that in certain cases might last hours, leading to economic losses and time-consuming tasks for the machine operator. Accordingly, a need is felt for providing solutions facilitating minimizing the downtime of the machine. The need is also felt to facilitate minimization of the downtime while also meeting elevate sterilization performance.

For example, when an electric arc occurs in case of electron beam emitters, the packaging machine operation has to be interrupted. An operator then manually cuts and extracts a lower portion of the packaging material, connects a cleaning pipe for cleaning the filling means, cleans the sterilization apparatus and repositions the packaging material in the correct position. A further sterilization is then performed before resuming the normal packaging machine operation. Such operations are time-consuming and the downtime may last several hours.

### DISCLOSURE OF INVENTION

It is therefore an object of the present invention to provide a sterilization apparatus to facilitate overcoming, in a straightforward and low-cost manner, at least one of the aforementioned drawbacks.

In particular, it is an object of the present invention to provide a sterilization apparatus able to react time-efficiently to an anomaly in operation.

It is a further object of the present invention to provide a packaging machine to facilitate overcoming, in a straightforward and low-cost manner, at least one of the aforementioned drawbacks. It is a further object of the present invention to provide a method for sterilizing to overcome, in a straightforward and low-cost manner, at least one of the aforementioned drawbacks.

According to the present invention, there is provided a sterilization apparatus as claimed in claim 1.

According to the present invention, there is also provided a packaging machine according to claim 12.

According to the present invention, there is also provided a method for sterilizing according to claim 15.

Preferred embodiments are claimed in the dependent claims.

### BRIEF DESCRIPTION OF THE DRAWINGS

A non-limiting embodiment of the present invention will be described by way of example with reference to the accompanying drawings, in which:

FIG. 1 is a schematic view of a packaging machine having a sterilization apparatus according to one or more embodiments, with parts removed for clarity;

FIG. 2 is a schematic view of a sterilization apparatus according to one or more embodiments, with parts removed for clarity; and

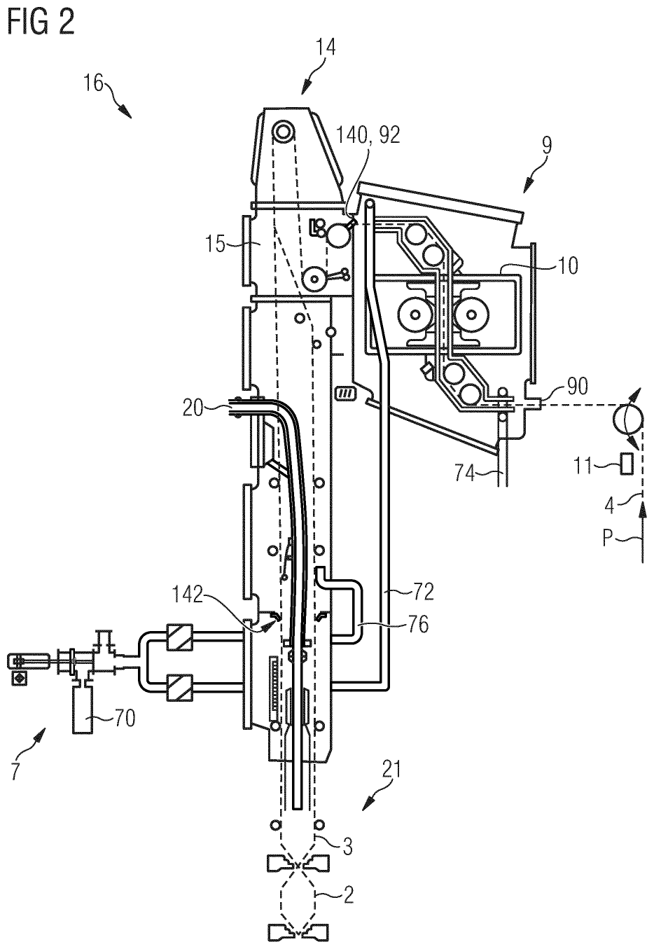

FIGS. 3 and 4 are a schematic representation of the sterilization apparatus 9 when an anomaly is detected and after interruption of web advancement, respectively.

### BEST MODES FOR CARRYING OUT THE INVENTION

Number 1 indicates as a whole a packaging machine for producing sealed packages 2 of a pourable product, in particular a pourable food product such as pasteurized milk, fruit juice, wine, tomato sauce, etc., from a tube 3 formed from a web 4 of packaging material. In particular, in use, tube 3 extends along a longitudinal axis, in particular having a vertical orientation.

Web 4 at least comprises a layer of fibrous material, in particular paper, covered on both sides with respective layers of heat-seal plastic material, e.g. polyethylene.

Preferably, web 4 also comprises a layer of gas- and light-barrier material, e.g. aluminum foil or ethylene vinyl alcohol (EVOH) film, and at least a first layer and a second layer of heat-seal plastic material. The layer of gas- and light-barrier material is superimposed on the first layer of heat-seal plastic material, and is in turn covered with the second layer of heat-seal plastic material. The second layer of heat-seal plastic material forms the inner face of package 2 eventually contacting the filled pourable food product.

More specifically, web 4 comprises a first face or surface 5 and a second face or surface 6, in particular first (sur)face 5 being the face of web 4 forming the inner face of the formed package 2 eventually contacting the filled pourable food product.

A typical package 2 obtained by packaging machine 1 comprises a longitudinal seam portion and a pair of transversal sealing bands, in particular a transversal top sealing band and a transversal bottom sealing band.

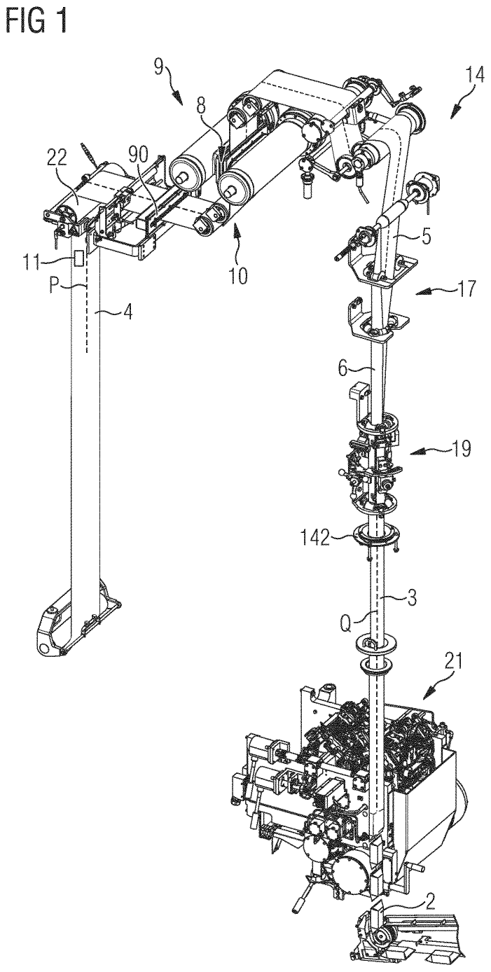

With particular reference to FIG. 1, packaging machine 1 is configured to advance web 4 along a web advancement path P, to sterilize web 4 during advancement along path P, to form tube 3 from web 4 and to fill tube 3 and to form single packages 2 from the filled tube 3.

Preferably, the packaging machine 1 comprises:
a sterilization apparatus 9 comprising a first sterilizing device 10 configured to sterilize at least the first (sur)face 5, preferentially also the second (sur)face 6, of the web 4,
an isolation chamber 14 Separating an inner environment 15, in particular an inner sterile environment, from an outer environment 16,
a tube forming device 17 arranged least partially, preferably fully, within isolation chamber 14 and being configured to form tube 3 from the, in use, advancing and sterilized web 4,
a sealing device 19 at least partially arranged within the isolation chamber 14 and being configured to longitudinally seal tube 3 formed by the tube forming device 17 so as to form a longitudinal seam portion of tube 3,
a filling unit 20 configured to fill tube 3 with the pourable product, in particular the pourable food product,
a package forming unit 21 configured to at least form and transversally seal tube 3, in particular the, in use, advancing tube 3, for forming packages 2, and
a plurality of conveyors configured to advance in a known manner web 4 along path P and to advance tube 3 along a tube advancement path Q.

In particular, the sterilization apparatus 9 may receive the web 4 of packaging material from a host station (not visible in the figures).

For example, the plurality of conveyors may be configured to advance the web 4 of packaging material from the host station to the sterilization apparatus 9 and/or from the sterilization apparatus 9 to the tube forming device 17. For example, the plurality of conveyors may be configured to advance the tube 3 from the tube forming device 17 to the sealing device 19 and/or from the sealing device 19 to the filling unit 20 and/or from the filling unit 20 to the package forming unit 21.

Preferably, the plurality of conveyors is adapted to advance tube 3 and any intermediate of tube 3 in a manner known as such along path Q, in particular from tube forming device 17 towards and at least partially through package forming unit 21.

In one or more embodiments, the plurality of conveyors comprises a first conveyor 22, (immediately) upstream of the sterilization apparatus 9. In particular, the first conveyor 22 may be positioned immediately upstream of an inlet 90 of the sterilization apparatus 9.

Preferentially, packaging machine 1 also comprises pressure control means configured to control the pressure within at least isolation chamber 14 and within at least portions of sterilization apparatus 9.

The sterilization apparatus 9 may be arranged upstream of the tube forming station 17, with respect to the web advancement path P.

The sterilization apparatus 9 may comprise an outlet 92. The outlet 92 may be positioned downstream with respect to the first sterilizing device 10 along the web advancing path P. The sterilized web 4 may advance through the outlet 92 towards the isolation chamber 14.

The isolation chamber 14 may comprise an inlet 140, wherein the inlet 140 of the isolation chamber 14 may be connected to the outlet 92 of the sterilization apparatus 9.

The isolation chamber 14 may comprise an outlet 142, wherein the tube 3, filled with pourable product, may exit the isolation chamber 14 at the outlet 142.

The package forming unit 21 may be arranged downstream of isolation chamber 14 and tube forming device 17 along path Q.

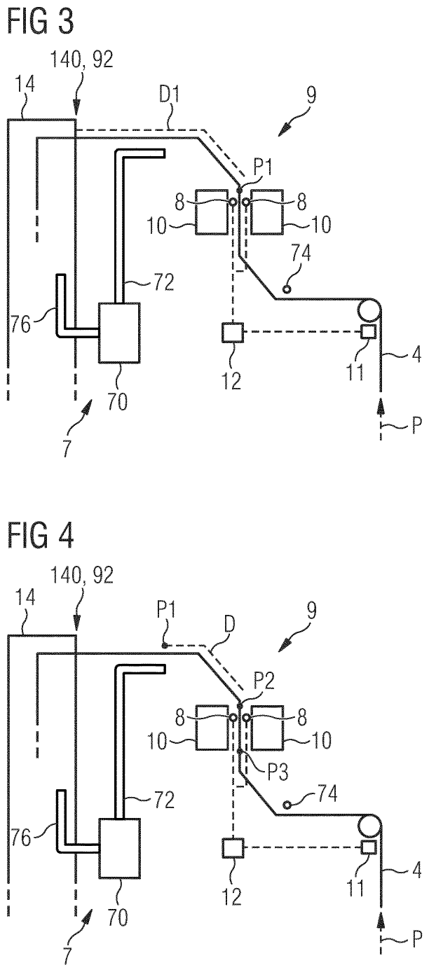

In one or more embodiments, as depicted with particular reference to FIGS. 3 and 4, the sterilization apparatus 9 comprises:
a first sterilizing device 10 configured to sterilize at least a first (sur)face 5, preferably also the second (sur)face 6, of the web 4 advancing through the first sterilizing device 10,
a second sterilizing device 7,
   a monitoring device 8, configured to monitor operation of the first sterilizing device 10,
a movement sensor 11 configured to monitor advancement of the web 4 along the advancement path P, and
a control unit 12, connected to the monitoring device 8 and the movement sensor 11, the control unit 12 configured to, if an anomaly (e.g. a deviation from standard operation, such as an electric arc event) is detected in operation of the first sterilizing device 10:
   interrupt advancement of the web 4,
   identify a distance D traveled by the web (4) between detection of the anomaly and interruption of advancement, and
   sterilize at least a portion P1, P2, P3 of the web 4 by means of the second sterilizing device 7, the portion P1, P2, P3 being associated (e.g. substantially corresponding) to the traveled distance D.

The first sterilizing device 10 may comprise an irradiation device configured to sterilize at least the first face 5, preferably also the second face 6, of the advancing web 4 by directing a sterilizing irradiation onto at least the first face 5 (and optionally second face 6).

A typical sterilization apparatus of this kind comprises an irradiation device typically having a pair of electron beam emitters spaced apart from one another. An advancement channel, through which, in use, the web 4 of packaging material advances, is interposed between the electron beam emitters. Each one of the electron beam emitters is configured to direct the respective electron beam onto one respective face of the web of packaging material advancing through the advancement channel.

A drawback of this type of sterilizing device 10 is that electric arc events may occur. During operation of the electron beam emitters an electric discharge, i.e. an electric arc, may occur that may change a dose of irradiation directed towards face 5 and/or face 6. That is, the anomaly may comprise an electric arc event. The control unit 12 may be configured to detect whether a change occurs in a dose of irradiation directed to the first face 5 (and optionally second face 6) of the web 4.

In the annexed figures, the first sterilizing device 10 is depicted as an irradiation device as described previously, however it will be appreciated that a different kind of first sterilizing device 10 may be employed, e.g. a hydrogen peroxide bath. In this case, the anomaly may comprise e.g. a change in a temperature of a peroxide bath.

When an anomaly occurs in the operation of the first sterilizing device 10, the control unit 12 may be configured to interrupt operation of the packaging machine 1. In particular, the control unit 12 may interrupt operation of the first sterilizing device 10, the tube forming device 17, the sealing device 19, the filling unit 20, the package forming unit 21 and/or the plurality of conveyors, preferably at the same time.

According to one or more embodiments, the traveled distance D is smaller than a distance D1 between the first sterilizing device 10 and the outlet 92. That is, the traveled distance D may be smaller than the distance D1 traveled by the web 4 between the first sterilizing device 10 and the outlet 92 during normal operation.

For example, the portion P1, P2, P3 being associated (e.g. substantially corresponding) to the traveled distance D may comprise the portion of the web 4 that passes at (e.g. through) the first sterilization device 10 between detection of the anomaly and interruption of advancement. For example, the portion P1, P2, P3 may include a first portion P1-P2 that exits the first sterilizing device 10 between detection of the anomaly and interruption of advancement and a second portion P2-P3 present at (e.g. in) the first sterilizing device 10 when the web advancement is interrupted.

Accordingly, the step of sterilizing at least the portion P1, P2, P3 associated to the traveled distance D may comprise sterilizing at least the first portion P1-P2 substantially corresponding to the distance D and/or sterilizing the second portion P2-P3 at the first sterilizing device 10 during interruption of web advancement.

Advantageously, the advancement of the web 4 may be interrupted before the first portion P1-P2 of the web 4 reaches the outlet 92 of the sterilization apparatus 9. This way the first portion P1-P2 does not proceed into the isolation chamber 14.

Advantageously, thus, the control unit 12 may be configured to (exactly and efficiently) identify the portion P1, P2, P3 that passes through the first sterilizing device 10 when a deviation from normal operation thereof is detected. As such, the portion P1, P2, P3 of the web 4 of packaging material that may be affected by the anomaly can be (accurately and precisely) identified.

For example, the movement sensor 11 may transmit a first sensor signal, e.g. indicative of speed of and/or position of and/or distance traveled by the web 4 of packaging material.

Advantageously, the web 4 of packaging material and the tube 3 present in the isolation chamber 14 and the sterilization apparatus 9 at the detection of the anomaly may be present (i.e. remain) in the packaging machine 1 before, during and after the sterilization by means of the second sterilizing device 7.

Advantageously, thanks to the identification of the traveled distance D and/or the portion P1, P2, P3 affected by the anomaly it is possible to significantly reduce the downtime due to such anomaly. The sterilization that ensues may be localized to the affected area and optionally to the neighboring area.

FIG. 3 depicts the sterilization apparatus 9 according to one or more embodiments when an anomaly occurs.

The control unit 12 may be configured to receive a second sensor signal from the monitoring device 8, e.g. indicative of sterilization of the web 4 of packaging material.

The control unit 12 may be configured to detect the anomaly if the second sensor signal reaches (and optionally exceeds) a predetermined threshold. For example, in case the monitoring device comprises an irradiation sensor, the second sensor signal may be indicative of an irradiation dose emitted by the first sterilizing device 10. The control unit 12 may be configured to detect the anomaly if the dose of emitted radiation reaches (e.g. becomes lower than) the threshold, e.g. a lower dose value of radiation.

Preferably, the control unit 12 may be configured to detect the anomaly when the second sensor signal reaches (or exceeds) the predetermined threshold for a predetermined time period. The predetermined time period may be between 50 ms and 120 ms, preferably between 70 and 110 ms, even more preferably 80 and 100 ms. The predetermined time period may be dependent on the advancement speed of the web 4 of packaging material. For example, the predetermined time period may be indirectly proportional to the advancement speed, i.e. if the advancement speed lowers the predetermined time period can increase.

Even more preferably, the control unit 12 may be configured to detect the anomaly when the second sensor signal continuously reaches (or exceeds) the predetermined threshold for the predetermined time period. That is, the second sensor signal reaches (or exceeds) the threshold for the entire predetermined time period. Advantageously, the predetermined time period may facilitate detecting real anomalies and filtering out false anomalies, e.g. fluctuations of the sensor signal due to electrical disturbances.

The control unit 12 may be configured to start identifying (e.g. calculating) the distance D at the beginning of the predetermined time period. The control unit 12 may be configured to interrupt advancement of the web 4 at the end of the predetermined time period.

FIG. 4 depicts the sterilization apparatus 9 according to one or more embodiments when operation of the sterilization apparatus 9 (and packaging machine 1) is interrupted. The first portion P1-P2 may (substantially) correspond to the traveled distance D. The second portion P2-P3 may comprise the portion present at (e.g. within) the first sterilizing device 10 during interruption of advancement.

In one or more embodiments, the second sterilization device 7 may comprise a hydrogen peroxide spray tank 70 configured to spray vaporized hydrogen peroxide at least at the portion P1, P2, P3 of the web 4 associated to the traveled distance D between detection of the anomaly and interruption.

As depicted in the figures, the spray tank 70 may be connected to a tube 72 configured to convey the vaporized hydrogen peroxide at the first sterilizing device 10, in particular at an outlet thereof with respect to the web advancement path P.

The spray tank 70 may be configured, by means of the tube 72, to sterilize the portion P1-P3 of the web 4. Air (e.g. gas flow) exiting the tube 72 may pass through the first sterilizing device 10 and may be collected through tube 74 (only partially shown). Such air may thus flow through the first sterilizing device in an opposite direction with respect to a web advancement direction along the web advancement path P.

The second sterilizing device 7 may be configured to sterilize the portion P1, P2, P3 of the web 4 as well as the first sterilizing device 10 and/or generally the sterilization apparatus 9.

As a preferential further precaution, the second sterilizing device 7 may be configured to sterilize the isolation chamber 14.

The spray tank 70 may be connected to a further tube 76 configured to convey the vaporized hydrogen peroxide at the isolation chamber 14, in particular the tube 76 may end downstream with respect to the tube forming device 17.

The spray tank 70 may be configured, by means of the further tube 76, to sterilize the isolation chamber 14. Air (e.g. gas flow) exiting the further tube 76 may pass through the isolation chamber 14, the first sterilizing device 10 and may be collected through tube 74.

In other words, when the web advancement is interrupted, the second sterilizing device 7 may be configured to spray sterilizing air comprising hydrogen peroxide the first sterilizing device 10 and preferably through the isolation chamber 14.

The second sterilizing device 7 can be actioned only if the web advancement is interrupted and/or the first sterilizing device 10 is deactivated.

According to one or more embodiments, the movement sensor 11 may comprise a rotative encoder, e.g. integrated within a conveyor, preferably upstream of the sterilization apparatus 9, in the plurality of conveyors configured to advance the web 4. Preferably, the first conveyor 22 may comprise the movement sensor 11, however any conveyor upstream of the sterilization apparatus 9 may comprise the movement sensor 11.

The conveyor comprising the movement sensor 11 may be free from actuating forces. In other words, the conveyor may include a free roller that rotates only thanks to the movement of the web 4. No external forces are applied to such conveyor for influencing the rotation thereof. This way, in the example, if the web 4 advances the first conveyor 22 rotates in a first direction, e.g. anti-clockwise in the figures, and if the web 4 recedes the first conveyor 22 rotates in a second direction opposite to the first direction, e.g. clockwise.

Advantageously, this may permit an even more accurate reading of the distance D, insofar as also possible backward movement of the web 4 of packaging material is taken into consideration.

A second conveyor may be in contact with the web 4 at the conveyor comprising the movement sensor 11, on an opposite side of the web 4 with respect to such conveyor comprising the movement sensor 11. This way, the web 4 of packaging material does not slide on the conveyors and a more accurate reading is performed.

According to one or more embodiments, one or more conveyors, e.g. (immediately) upstream of the sterilization apparatus 9, may comprise an actuator configured to control a brake velocity of the advancing web 4. Advantageously, this way the distance D traveled before interruption of the web 4 may be reduced.

In addition or in alternative, the actuator may be configured to control a tensioning of the advancing web 4. Advantageously, this way the web 4 of packaging material is kept in tension also when the packaging machine 1 stops, which facilitates sterilization and reduces a risk of damages on the web 4.

In one or more embodiments, the first conveyor 22 may comprise the movement sensor 11 and the actuator. In addition or in alternative, further conveyor(s) e.g. (immediately) upstream of the first conveyor 22 may comprise the actuator.

The control unit 12 may be configured to resume advancement of the web 4 of packaging material after the step of sterilizing by means of the second sterilizing device 7 is completed. That is, before, during and after the anomaly detection the web 4 of packaging material remains within the packaging machine 1.

At least the packages 2 formed with the packaging material present within the sterilization apparatus 9 and isolation chamber 14 during the step of sterilizing by means of the second sterilizing device 7 may be discarded.

According to one more embodiments, the control unit 12 may be configured to transmit an alarm signal, e.g. to an user interface, if the traveled distance D is greater than a predetermined distance, e.g. the distance D1 between the first sterilizing device 10 and the outlet 92 of the sterilization apparatus 9. A cleaning and resterilization process is then performed to the isolation chamber 14, filling unit 20 and sterilization apparatus 9.

One or more embodiments may relate to a method of sterilizing a web 4 of packaging material performed by a sterilization apparatus 9 according to one or more embodiments. The method may comprise:
advancing the web 4 of packaging material along a web advancement path P,
sterilizing at least a first face 5, preferably also a second face 6, of the advancing web 4 by means of a first sterilizing device 10,
monitoring operation of the first sterilizing device 10,
monitoring advancement of the web 4 along the advancement path P, and
if an anomaly is detected in operation of the first sterilizing device 10:
   interrupting advancement of the web 4,
   identifying a distance D traveled by the web 4 between detection of the anomaly and interruption of advancement, and
   sterilizing at least a portion P1, P2, P3 of the web 4 by means of the second sterilizing device 7, the portion P1, P2, P3 of the web being associated to the traveled distance D.

The advantages of sterilization apparatus 9 according to the present invention will be clear from the foregoing description. In particular, the sterilization apparatus 9 may minimize operative downtimes of the packaging machine 1 with respect to what is known in the art. Even more in particular, the operative downtime may be reduced from several hours to tens of minutes.

Clearly, changes may be made to sterilization apparatus 9 as described herein without, however, departing from the scope of protection as defined in the accompanying claims.

1. A sterilization apparatus for sterilizing a web of packaging material advancing along an advancement path, the sterilization apparatus comprising:
a first sterilizing device configured to sterilize at least a first face the web advancing through the first sterilizing device,
a second sterilizing device,
a monitoring device, configured to monitor operation of the first sterilizing device,
a movement sensor configured to monitor advancement of the web along said advancement path, and
a control unit, connected to the monitoring device and the movement sensor, the control unit configured to, if an anomaly is detected in operation of the first sterilizing device:
interrupt advancement of the web,
identify a distance traveled by the web between detection of the anomaly and interruption of advancement, and
sterilize at least a portion of the web by means of the second sterilizing device, the portion of the web being associated to said traveled distance.

2. The sterilization apparatus according to claim 1, comprising an outlet and wherein said traveled distance is smaller than a distance between said first sterilizing device and said outlet.

3. The sterilization apparatus according to claim 1, wherein the step of detecting an anomaly in operation of the first sterilizing device comprises:
receiving a sensor signal from the monitoring device, and
checking whether the sensor signal reaches a predetermined threshold for a predetermined time period, preferably continuously.

4. The sterilization apparatus according to claim 1, wherein the first sterilization device comprises an irradiation device configured to sterilize at least the first face of the advancing web by directing a sterilizing irradiation onto at least the first face and wherein the anomaly is an electric arc event.

5. The sterilization apparatus according to claim 4, wherein the monitoring device comprises an irradiation sensor configured to detect a dose of radiation emitted by the first sterilization device and wherein the control unit is configured to detect an anomaly if the dose of radiation emitted reaches a predetermined threshold.

6. The sterilization apparatus according to claim 1, wherein the second sterilization device comprises a hydrogen peroxide spray tank configured to spray vaporized hydrogen peroxide at least at said portion of the web.

7. The sterilization apparatus according to claim 1, wherein the movement sensor comprises a rotative encoder integrated within a conveyor configured to advance the web.

8. The sterilization apparatus according to claim 7, wherein the conveyor is free from actuating forces.

9. The sterilization apparatus according to claim 1, wherein the control unit is configured to transmit an alarm signal, if said traveled distance is greater than a predetermined distance,
wherein preferably the predetermined distance corresponds to a distance between the first sterilizing device and an outlet of the sterilization apparatus.

10. The sterilization apparatus according to claim 1, wherein the control unit is configured to resume advancement of the web of packaging material after the step of sterilizing by means of the second sterilizing device is completed.

11. The sterilization apparatus according to claim 1, wherein the second sterilizing device is configured to sterilize said portion of the web and said first sterilizing device.

12. A packaging machine for producing sealed packages of a pourable product from a web of packaging material, the packaging machine comprising at least:
a sterilization apparatus according to claim 1,
an isolation chamber connected to the sterilization apparatus and separating an inner environment from an outer environment,
a tube forming device at least partially arranged within the isolation chamber and being configured to form a tube from the web of packaging
a sealing device at least partially arranged within the isolation chamber and being configured to longitudinally seal the tube formed by the tube forming device,
a filling unit configured to fill the tube with the pourable product,
a package forming unit configured to form and to transversally seal the tube for forming the packages, and
a plurality of conveyors configured to advance the web of packaging material along a web advancement path and for advancing the tube along a tube advancement path.

13. The packaging machine according to claim 12, comprising a conveyor upstream of the sterilization apparatus, wherein the movement sensor comprises a rotative encoder integrated within the conveyor.

14. The packaging machine according to claim 12, wherein the plurality of conveyors comprises at least one conveyor, upstream of the sterilization apparatus, comprising an actuator configured to control a brake velocity of the advancing web and/or control a tensioning of said advancing web.

15. A method of sterilizing a web of packaging material comprising the steps of:
advancing the web of packaging material along a web advancement path,
sterilizing at least a first face of the advancing web means of a first sterilizing device,
monitoring operation of the first sterilizing device,
monitoring advancement of the web along said advancement path, and
if an anomaly is detected in operation of the first sterilizing device:
interrupting advancement of the web,
identifying a distance traveled by the web between detection of the anomaly and interruption of advancement, and
sterilizing at least a portion of the web by means of the second sterilizing device, the portion of the web being associated to said traveled distance.
